(19) United States
(12) Patent Application Publication
(10) Pub. No.: US 2015/0151141 A1
(43) Pub. Date: Jun. 4, 2015

(54) Device and Method for Focusing Pulses

(71) Applicants: Bastien Arnal, Paris (FR); Mathieu Pernot, Paris (FR); Mickaël Tanter, Bagneux (FR); Mathias Fink, Meudon (FR)

(72) Inventors: Bastien Arnal, Paris (FR); Mathieu Pernot, Paris (FR); Mickaël Tanter, Bagneux (FR); Mathias Fink, Meudon (FR)

(73) Assignees: Centre National De La Recherche Scientifique - CNRS, Paris Cedex 16 (FR); INSERM Instititut National de la Sante et de la Recherche Medicale, Paris (FR)

(21) Appl. No.: 14/406,125

(22) PCT Filed: Jun. 4, 2013
(86) PCT No.: PCT/FR2013/051259

§ 371 Date: Dec. 5, 2014

(30) Foreign Application Priority Data
Jun. 6, 2012 (FR) 12 55251

(51) Int. Cl. A61N 7/00; G10K 15/00

(52) U.S. Cl. CPC A61N 7/00; G10K 15/00; A61N 2007/006

(57) ABSTRACT

Device for focusing pulses comprising at least emitting means comprising a network of transducers, these emitting means being adapted to make the network of transducers emit, into a reflective cavity, at least one wave focused onto at least one target point of a target medium. The reflective cavity comprises a multi-scattering medium adapted to cause multiple scattering of said wave.

The present invention relates to methods and devices for focusing waves. More specifically, it relates to methods and devices for generating high intensity waves at a target point of a target medium, such as acoustic waves for medical applications.

The invention therefore relates to a focusing device comprising at least pulse emitting means comprising a transducer array (also referred to herein as a transducer network), the emitting means being adapted cause the transducer array to emit, into a reflective cavity, at least one wave focused on at least one target point of a target medium.

There are known devices for emitting waves, for example devices emitting high-intensity focused ultrasound (HIFU) waves or lithotripsy devices. These devices have disadvantages because their focal point cannot be moved quickly and over long distances by simple means.

Document US 2009/0216128 discloses an example of a device seeking to solve this problem. The device comprises a reflective cavity with a randomly uneven surface in which it is possible to generate and control waves having a movable focal point. The cavity is filled with water and provided with a window placed in contact with the target area in order to enhance the transmission of acoustic waves to the target area.

This solution has drawbacks, however. The cavity forms a reverberator with a low quality factor and significant losses. The intensity of the wave at the target point is low.

The present invention is intended to overcome these disadvantages.

For this purpose, according to the invention, a device for focusing pulses of the type in question is characterized in that the reflective cavity comprises a multi-scattering medium adapted to cause a multiple scattering of said wave.

With these arrangements, the quality factor of the reverberator formed by the cavity remains significant while a high transmittance is maintained between the cavity and the environment. These two features render the device able to generate high-intensity pulses and/or waves in the environment. This multi-scattering medium can be considered an effective medium with adjustable transmission coefficient. The position of the target point is easily movable over a large volume. Losses by the reverberator formed by the cavity are low and the characteristics of this reverberator are easily adjustable through the choice of multi-scattering medium. The transducers used can be low power and generate high intensity waves at the target point, due to the high quality factor of the reverberator. The number of transducers used can be reduced due to the generation of virtual sources.

In preferred embodiments of the device, one or more of the following arrangements may possibly be used:
the multi-scattering medium comprises a plurality of scatterers;
the scatterers are substantially identical to each other;
each scatterer has at least one transverse dimension substantially between 0.1 and 5 times the wavelength of the wave in the reflective cavity;
each scatterer has at least one transverse dimension substantially between 0.5 and 1 times the wavelength of the wave in the reflective cavity;
the scatterers are distributed within the multi-scattering medium in a non-periodic manner;
the scatterers are distributed within the multi-scattering medium so that their surface density in a cross-section of the reflective cavity is substantially between 2 and 30 scatterers per surface area equivalent to a square having a side equal to ten times the wavelength of the wave in the reflective cavity;
the acoustic scatterers are distributed within the multi-scattering medium in such a way that their volume packing density is between 1% and 30%;
each acoustic scatterer has a ratio of length to width that is greater than 5;
the wave is an acoustic wave;
the reflective cavity contains a liquid;
the reflective cavity comprises a window in at least one of its ends;
the multi-scattering medium is placed near said end;
the target medium comprises living tissue;

the device further comprises a lens placed between the reflective cavity and the target medium;
the emitting means are adapted to emit the wave s(t) toward a number K, equal to at least 1, of predetermined target points k within the target medium, by causing each transducer i of the array to emit an emission signal:

$${s}_{i} ( t ) = {\sum }_{k = 1}^{K} {e}_{ik} ( t ) ⊗ s ( t )$$

where the signals eᵢₖ(t) are predetermined individual emission signals adapted so that when the transducers i emit signals eᵢₖ(t), a pulse wave is generated at target point k;
the emitting means are adapted to emit a wave capable of generating cavitation bubbles at a target point.

The invention also relates to a method for focusing pulses, comprising at least one emission step during which an array of transducers emits at least one wave focused on least one target point of a target medium, and said wave travels through a reflective cavity before reaching the target medium, the method being characterized in that during the emission step, a multiple scattering of said wave is caused by a multi-scattering medium located in the reflective cavity.

In preferred embodiments of the method, one or more of the following arrangements may possibly be used:
during the emission step, the wave s(t) is emitted towards a number K, at least equal to 1, of predetermined target points k within the target medium, by causing each transducer i of the array to emit an emission signal:

$${s}_{i} ( t ) = {\sum }_{k = 1}^{K} {e}_{ik} ( t ) ⊗ s ( t )$$

where the signals eᵢₖ(t) are predetermined individual emission signals adapted so that when the transducers i emit signals eᵢₖ(t), a pulse wave is generated at target point k;
the signals eᵢₖ(t) are each encoded into between 1 and 64 bits;
the signals eᵢₖ(t) are each encoded into 1 bit;
the individual emission signals eᵢₖ(t) are determined experimentally during a learning step, prior to said emission step;
during the learning step, an ultrasonic pulse signal is successively emitted at each predetermined target point k, the signals rᵢₖ(t) received by each transducer i of the array from the emission of said ultrasonic pulse signal are captured, and the individual emission signals eᵢₖ(t) are determined by time reversal of the received signals rᵢₖ(t):

eᵢₖ(t)=rᵢₖ(−t);
during the learning step, a liquid medium distinct from the target medium is placed in contact with the reflective cavity, and said pulse signal is emitted out from said liquid medium;
for a predetermined target point k, an ultrasonic pulse signal is successively emitted at each transducer i of the array, the signals rᵢₖ(t) received at target point k from the emission of said ultrasonic pulse signal are captured, and the individual emission signals eᵢₖ(t) are determined by time reversal of the received signals rᵢₖ(t):

eᵢₖ(t)=rᵢₖ(−t);
during the learning step, a liquid medium distinct from the target medium is placed in contact with the reflective cavity, and the signals rᵢₖ(t) are captured in said liquid medium;
the liquid medium used during the learning step essentially comprises water, and during the emission step the target medium in which the wave is focused comprises living tissue;
the individual emission signals eᵢₖ(t) are determined by calculation;
during the emission step, a wave capable of generating cavitation bubbles at the target point is emitted;
the wave is an acoustic wave;
the emission step is repeated at least once at a rate of between 10 Hz and 1000 Hz.

Other features and advantages of the invention will be apparent from the following description of one of its embodiments, given by way of non-limiting example and with reference to the accompanying drawing.

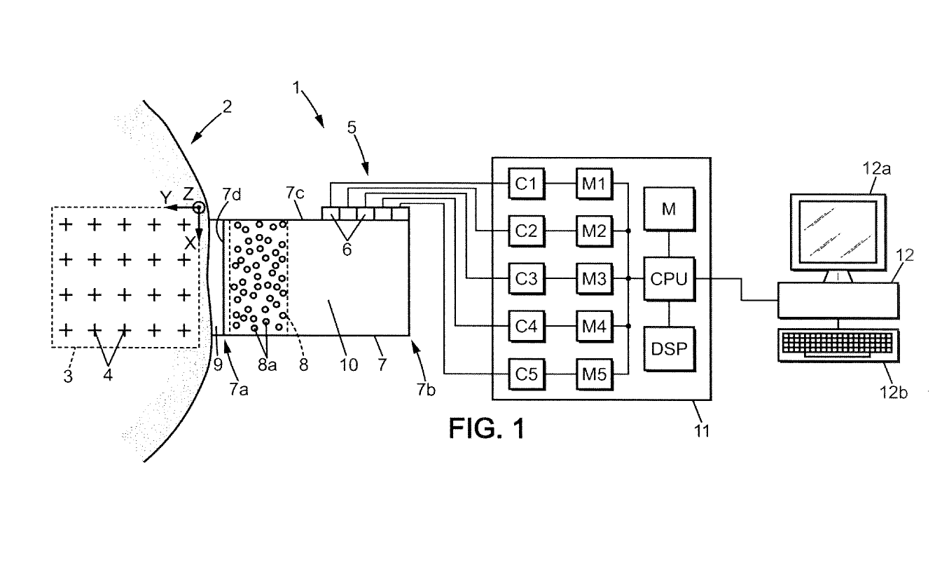

In the drawing, FIG. 1 is a schematic view illustrating a pulse focusing device according to an embodiment of the invention, for example an acoustic pulse focusing device.

According to some embodiments of the invention, the waves and pulses mentioned may be acoustic, optical, or electromagnetic waves and/or pulses.

The electromagnetic waves and/or pulses are, for example, waves and/or pulses in the radio frequency or terahertz region, for example having a center frequency of between a few megahertz and a few terahertz.

The sound waves may be ultrasound waves for example, such as waves and/or pulses having a center frequency of between 200 kHz and 100 MHz, for example between 0.5 MHz and 10 MHz.

All elements of the pulse focusing device 1 are selected and adapted by those skilled in the art according to the type and frequency of the waves and/or pulses in question.

For example, the emission and reception elements, the transmission windows, the reflective cavity and other reflective elements, the scattering medium and scatterers, the lenses and focusing elements, and any other element used in the pulse focusing device 1 and in the focusing method are respectively adapted to the type and frequency of the waves and/or pulses selected by the skilled artisan.

The pulse focusing device 1 represented in FIG. 1 is intended for example to focus the pulses in a target medium 2, for example living tissue which may be part of a patient's body in histotripsy applications, or a part of an industrial object in industrial applications, or some other target medium.

More specifically, the pulse focusing device 1 is intended for focusing pulses in a target area 3 within the target medium 2, this area 3 possibly being three-dimensional.

For this purpose, the device 1 is adapted to emit waves focused on one or more predetermined target points 4 within the target area 3.

The waves are emitted by the emission and reception elements, for example an array 5 of transducers 6, which are placed in or attached to a reflective cavity 7.

There can be any number of transducers 6, ranging from 1 to several hundred, for example several tens of transducers.

The array 5 may be a linear array, with the transducers side by side along a longitudinal axis of the array as can be seen in known ultrasound probes.

The array 5 may be a two-dimensional array so as to emit three-dimensional focused waves.

The reflective cavity 7 may be filled with a liquid 10, for example water.

The reflective cavity 7 may be filled with a gas, for example a gas having a low capacity for absorbing the waves and/or pulses generated by the transducers 6.

The reflective cavity comprises walls made of a material forming a highly reflective interface for the waves. The walls of the reflective cavity 7 may, for example, be made of a metal plate, an electromagnetic or optical mirror, or a thin film separating the liquid contained in the cavity from the air outside the cavity so as to create a highly reflective liquid-air interface for acoustic waves and/or pulses.

The reflective cavity 7 is in contact at one of its ends 7a with the target medium 2, directly or through a lens 9, for example an acoustic, optical, or electromagnetic lens. It may, for example, be provided with a window 7b at said end 7a, the window 7b having a wall that transmits the waves with little loss.

The reflective cavity 7 can have the general shape of a rectangular parallelepiped, the transducers 6 of the array being for example located on or near an end 7b of the reflective cavity 7 which is located opposite the end 7a in contact with the target medium 2.

The reflective cavity may be generally cylindrical in shape, for example a right circular cylinder or some other type of cylinder, extending along a cavity extension direction Y and having a flat face on the side opposite the end 7a in contact with the target medium 2.

In another embodiment, the reflective cavity 7 may have an irregular shape, for example with recesses or protuberances formed in its walls.

The reflective cavity 7 further comprises a multi-scattering medium 8 adapted to be traversed by the wave before it reaches the target medium 2, and to cause multiple scattering of the wave.

The multi-scattering medium 8 may be located, for example, near the end 7a of the reflective cavity 7 in contact with the target medium 2.

The multi-scattering medium 8 may cover, for example, an entire cross-section of the reflective cavity 7, taken perpendicular to the cavity extension direction Y.

The multi-scattering medium 8 may comprise any number of scatterers 8a, ranging from several tens to several thousands, for example several hundred.

The scatterers 8a are adapted to scatter the acoustic wave.

The scatterers 8a are advantageously distributed randomly or non-periodically in the multi-scattering medium, meaning that their distribution does not exhibit a periodic structure.

In the example in FIG. 1, they have the general shape of a vertical rod extending along an extension direction Z from a lower end to an upper end.

The extension directions of the acoustic scatterers 8a may for example be parallel to each other and perpendicular to the longitudinal axis of the transducer array and to the cavity extension direction Y.

The scatterers may be held in place by frames or be attached to the walls of the reflective cavity 7 at their ends.

Alternatively, they may take the form of beads, granules, cylinders, or any three-dimensional solid, and be held in place by a foam, an elastomer, or three-dimensional frames so that they are distributed over all three dimensions of the space and form the multi-scattering medium 8.

The shape and density of the scatterers 8a and the dimensions of the multi-scattering medium 8 are chosen to ensure maximum multiple scattering of the wave as well as good transmission.

The scatterers 8a may have a surface that is highly reflective for the wave, for example a metal, an optical or electromagnetic mirror, or a surface having a significant difference in impedance compared to the medium of the reflective cavity.

The scatterers 8a may, for example, have a transverse cross-section that is substantially between 0.1 and 5 times the wavelength of the wave in the reflective cavity, for example between 0.5 and 1 times said wavelength.

Said transverse cross-section is understood to be a cross-section taken perpendicularly to their extension direction, for example perpendicularly to their longest extension direction.

Thus, the scattering mean free path (the average distance between two scattering events of the wave) can be minimized, and the transport mean free path (the average distance after which the wave loses its initial direction) can be maximized. By way of non-limiting example, for an acoustic wave having a center frequency of about 1 MHz, the scatterers 8a can for example have a transverse cross-section, taken perpendicularly to their extension direction or along their smallest transverse cross-section, contained within a circle approximately 0.8 mm in diameter, and a length of 9 cm, for example along their extension direction.

Similarly, the scatterers 8a can be distributed in the multi-scattering medium 8 so that their surface density in a transverse cross-section of the multi-scattering medium 8 is substantially between 2 and 30 scatterers per surface area equivalent to a square having a side equal to ten times the wavelength of the wave in the reflective cavity 7.

Said transverse cross-section is understood to be a cross-section taken perpendicularly to the extension direction of the scatterers 8a and/or to the longest extension direction of the multi-scattering medium 8.

Again by way of example, the scatterers 8a can be distributed within the multi-scattering medium 8 so that their surface density in a cross-section of the multi-scattering medium 8 transverse to the extension direction Z of the scatterers 8a, is, for an acoustic wave having a center frequency of about 1 MHz, ten or so scatterers 8a per square centimeter, for example eighteen acoustic scatterers 8a per square centimeter.

In the case of a three-dimensional multi-scattering medium, the scatterers 8a can be distributed in the multi-scattering medium 8 so that their volume packing density within the multi-scattering medium 8 is between 1% and 30%.

Finally, the length of the multi-scattering medium 8, along the direction of propagation of the wave, may be a few centimeters, for example two centimeters for an acoustic wave.

In the case of a three-dimensional multi-scattering medium 8, the volume packing density of the scatterers 8a could be, for example, ten or so scatterers 8a per cubic centimeter and the dimensions of the multi-scattering medium 8 along the three spatial directions could be a few centimeters.

Of course, other general forms for the reflective cavity 7, the multi-scattering medium 8, and/or the scatterers 8a can be considered.

A lens 9 can also be placed between the target medium 4 and the reflective cavity 7.

Depending on the embodiment of the invention, the lens 9 may be an acoustic, optic, or electromagnetic lens adapted to focus the waves and/or pulses in one or two directions.

In some embodiments, the reflective cavity 7 and the multi-scattering medium 8 may be adapted to form a reverberator with a high quality factor.

In an embodiment where the wave is an acoustic wave, the pressure of the acoustic wave generated by the transducer array can thus be amplified by more than 20 dB by the reverberator formed by the reflective cavity 7 and the multi-scatterer medium 8.

In an embodiment where the wave is an optical or electromagnetic wave, the power of the pulse generated at the focal point will also be strongly amplified.

The transducers 6 of the array may be placed on a face of the reflective cavity 7 opposite the target medium 2 or on a side face of the cavity 7c.

Alternatively, they may be placed on a side face 7c and oriented so as to emit waves toward the multi-scattering medium, at a certain angle relative to the cavity extension direction Y, for example 60°.

The transducers 6 are controlled independently of each other by a microcomputer 12 (typically with user interfaces such as a display 12a and a keyboard 12b), possibly by means of a processor CPU and/or a graphics processing unit GPU contained for example in a cabinet 11 connected by a flexible cable to the transducers 6.

This cabinet 11 may comprise for example:
an analog-to-digital converter C1-C5 connected to each transducer 6;
memory M1-M6 connected to the analog-to-digital converter of each transducer 6 and to the processor CPU and/or graphics processing unit GPU;
and general memory M connected to the processor CPU.

The device may also include a digital signal processor or “DSP” connected to the processor CPU.

The device described above operates as follows.

Prior to any focusing operation, a matrix of individual emission signals eᵢₖ(t) is determined such that, to generate a wave s(t) at a target point k, each transducer i of the array 5 emits an emission signal:

Sᵢ(t)=eᵢₖ(t) [symbol] s(t).

These individual emission signals may possibly be determined by calculation (for example using a spatio-temporal inverse filter method), or may be determined experimentally during a preliminary learning step.

During this learning step, it is advantageous to have an ultrasonic pulse signal emitted by an emitter such as a hydrophone, successively at each target point k, and the signals rᵢₖ(t) received by each transducer i of the array 5 from the emission of said ultrasonic pulse signal are captured. The signals rᵢₖ(t) are converted by the analog-to-digital converters and stored in the memory connected to the processor CPU, which then calculates the individual emission signals eᵢₖ(t) by time reversal of said received signals:

eᵢₖ(t)=rᵢₖ(−t).

If the target medium 2 is a liquid medium, it may optionally be possible to perform the preliminary learning step by successively positioning the ultrasonic wave emitter at the different target points 4 of the target area 3. If the medium 2 is living tissue, for example a body part of a patient or a similar medium comprising a large amount of water, it may be possible to carry out the learning phase by replacing the medium 2 with a volume of liquid preferably consisting mostly of water, successively positioning the ultrasonic wave emitter at the locations of the various target points 4 identified with respect to the reflective cavity 7.

By making use of the principle of reciprocal space, we can also determine the signals eᵢₖ(t) by successively placing one or more hydrophones at the target points k in said liquid medium. For each position k of the hydrophone, an ultrasonic pulse is successively emitted by each transducer i, and the signals rᵢₖ(t) are captured by the hydrophone. The signals eᵢₖ(t) are then determined by time reversal:

eᵢₖ(t)=rᵢₖ(−t).

When one or more waves are then to be focused on a predetermined target point k within the target area 3, the reflective cavity 7 is placed in contact with the target medium, and an emission signal is emitted by each transducer i of the array:

sᵢ(t)=eᵢₖ(t) [symbol] s(t).

Alternatively, it is equally possible to generate a wave s(t) focused on a number K, greater than 1, of target points 4 in the target area 3, by having each transducer i of the array 5 emit an emission signal

$${s}_{i} ( t ) = {\sum }_{k = 1}^{K} {e}_{ik} ( t ) ⊗ s ( t ) .$$

The waves thus emitted by the transducers 6 of the array have a center frequency that can be between 200 kHz and 100 MHz, for example between 0.5 MHz and 10 MHz.

Moreover, the emission step can be repeated at a rate of between 10 Hz and 1000 Hz.

In one embodiment making use of acoustic waves, cavitation bubbles can be generated at the target point 4. To do this, a negative pressure above the cavitation threshold, for example −15 MPa, can be generated at the target point 4 by emitting an ultrasonic acoustic wave s(t) (continuously or non-continuously).

Although the device 1 has been described above as a pulse focusing device, it is possible to use the device, in addition to or independently of the focusing, for imaging such as ultrasonic imaging as will now be described.

When performing imaging, for example ultrasonic imaging, after each emission of an acoustic wave focused on one or more target points 4 of the target area 3, the echoes emitted by the target medium 2 are captured by means of the transducers 6 of the array. The captured signals are digitized by samplers C1-C5 and stored in memory M1-M6, and then processed by a conventional beamforming technique which performs focusing at reception on the target point or points 4 aimed for at emission.

The processing in question, which includes imposing different delays on the captured signals and capturing these signals, can be implemented by a summation circuit S connected to the memory M1-M6 or to the CPU. Advantageously, during this echo reception step, one can take advantage of the nonlinear behavior of at least one of the elements traversed by the wave, in other words the target medium 2, the reflective cavity 7, and/or the multi-scattering medium 8 (in practice, it is primarily the target medium 2 that will present non-linear behavior, the reflective cavity 7 and the multi-scattering medium 8 preferably having linear behavior). The wave is generated with sufficient amplitude to generate harmonics of the center frequency fc of the wave, at a sufficient level to be able to listen for the echoes returning from the target medium 2 at a listening frequency that is an integer multiple of the center frequency fc of the emission.

Advantageously, one thus listens for echoes returning from the target medium 2 at a frequency that is two or three times the frequency fc.

This selective listening frequency can be obtained either through the composition of the transducers 6, in a known manner, or by frequency filtering the signals from the transducers 6.

Due to this listening at a frequency different from the frequency fc, this eliminates any listening interference from the wave itself.

Note that the method and the device according to the invention would also be useful for precision ultrasonic cleaning applications or ultrasonic welding.

1. A device for focusing pulses, comprising at least emitting means comprising an array of transducers, said emitting means being adapted to cause the transducer array to emit, into a reflective cavity, at least one wave focused on at least one target point of a target medium,
wherein the reflective cavity comprises a multi-scattering medium adapted to cause a multiple scattering of said wave.

2. The device device according to claim 1, wherein the multi-scattering medium comprises a plurality of scatterers.

3. The device according to claim 2, wherein the scatterers are substantially identical to each other.

4. The device according to claim 2, wherein each scatterer has at least one transverse dimension substantially between 0.1 and 5 times the wavelength of the wave in the reflective cavity.

5. The device according to claim 2, wherein each scatterer has at least one transverse dimension substantially between 0.5 and 1 times the wavelength of the wave in the reflective cavity.

6. The device according to claim 2, wherein the scatterers are distributed within the multi-scattering medium in a non-periodic manner.

7. The device according to claim 2, wherein the scatterers are distributed within the multi-scattering medium so that their surface density in a cross-section of the reflective cavity is substantially between 2 and 30 scatterers per surface area equivalent to a square having a side equal to ten times the wavelength of the wave in the reflective cavity.

8. The device according to claim 2, wherein the acoustic scatterers are distributed within the multi-scattering medium in such a way that their volume packing density is between 1% and 30%.

9. The device according to claim 2, wherein each acoustic scatterer has a ratio of length to width that is greater than 5.

10. The device according to claim 1, wherein the wave is an acoustic wave.

11. The device according to claim 1, wherein the reflective cavity contains a liquid.

12. The device according to claim 1, wherein the reflective cavity comprises a window in at least one of its ends.

13. The device according to claim 12, wherein the multi-scattering medium is placed near said end.

14. The device according to claim 1, wherein the target medium comprises living tissue.

15. The device according to claim 1, comprising a lens placed between the reflective cavity and the target medium.

16. The device according to claim 1, wherein the emitting means are adapted to emit the wave s(t) toward a number K, equal to at least 1, of predetermined target points k within the target medium, by causing each transducer i of the array to emit an emission signal:
$${s}_{i} ( t ) = {\sum }_{k = 1}^{K} {e}_{ik} ( t ) ⊗ s ( t )$$
where the signals eᵢₖ(t) are predetermined individual emission signals adapted so that when the transducers i emit signals eᵢₖ(t), a pulse wave is generated at target point k.

17. The device according to claim 10, wherein the emitting means are adapted to emit a wave capable of generating cavitation bubbles at a target point.

18. A method for focusing pulses, comprising at least one emission step during which an array of transducers emits at least one wave focused on at least one target point of a target medium, and said wave travels through a reflective cavity before reaching the target medium,
wherein in that during the emission step a multiple scattering of said wave is caused by a multi-diffusing medium located in the reflective cavity.

19. The method according to claim 18, wherein, during the emission step, the wave s(t) is emitted towards a number K, at least equal to 1, of predetermined target points k within the target medium, by causing each transducer i of the array to emit an emission signal:
$${s}_{i} ( t ) = {\sum }_{k = 1}^{K} {e}_{ik} ( t ) ⊗ s ( t )$$
where the signals eᵢₖ(t) are predetermined individual emission signals adapted so that when the transducers i emit signals eᵢₖ(t), a pulse wave is generated at target point k.

20. The method according to claim 19, wherein the signals eᵢₖ(t) are each encoded into between 1 and 64 bits.

21. The method according to claim 20, wherein the signals eᵢₖ(t) are each encoded into 1 bit.

22. The method according to claim 19, wherein the individual emission signals eᵢₖ(t) are determined experimentally during a learning step, prior to said emission step.

23. The method according to claim 22, wherein, during the learning step, an ultrasonic pulse signal is successively emitted at each predetermined target point k, the signals rᵢₖ(t) received by each transducer i of the array from the emission of said ultrasonic pulse signal are captured, and the individual emission signals eᵢₖ(t) are determined by time reversal of the received signals rᵢₖ(t):
eᵢₖ(t)=rᵢₖ(−t).

24. The method according to claim 22, wherein, during the learning step, a liquid medium distinct from the target medium is placed in contact with the reflective cavity, and said pulse signal is emitted out from said liquid medium.

25. The method according to claim 22, wherein, during the learning stage, for a predetermined target point k, an ultrasonic pulse signal is successively emitted at each transducer i of the array, the signals rᵢₖ(t) received at target point k from the emission ion of said ultrasonic pulse signal are captured, and the individual emission signals eᵢₖ(t) are determined by time reversal of the received signals rᵢₖ(t):
eᵢₖ(t)=rᵢₖ(−t).

26. The method according to claim 25, wherein, during the learning step, a liquid medium distinct from the target medium is placed in contact with the reflective cavity, and the signals rᵢₖ(t) are captured in said liquid medium.

27. The method according to claim 26, wherein the liquid medium used during the learning step essentially comprises water, and during the emission step the target medium in which the wave is focused comprises living tissue.

28. The method according to claim 19, wherein the individual emission signals eᵢₖ(t) are determined by calculation.

29. The method according to claim 18, wherein, during the emission step, a wave capable of generating cavitation bubbles at the target point is emitted.

30. The method according to claim 18, wherein the wave is an acoustic wave.

31. The method according to claim 18, wherein the emission step is repeated at least once at a rate of between 10 Hz and 1000 Hz.
